(19) United States
(12) Patent Application Publication
(10) Pub. No.: US 2022/0330530 A1
(43) Pub. Date: Oct. 20, 2022

(54) METHOD OF MAKING HUMAN MOUSE XENOGRAFTS

(71) Applicant: The University of North Carolina at Chapel Hill, Chapel Hill, NC (US)

(72) Inventors: J. Victor Garcia-Martinez, Durham, NC (US); Angela Wahl, Chapel Hill, NC (US); Orrin Thayer, Chapel Hill, NC (US)

(21) Appl. No.: 17/642,768

(22) PCT Filed: Sep. 11, 2020
(86) PCT No.: PCT/US2020/050447

§ 371 Date: Mar. 14, 2022

Related U.S. Application Data
(60) Provisional application No. 62/899,995, filed on Sep. 13, 2019.

(51) Int. Cl. A01K 67/027

(52) U.S. Cl. CPC A01K 67/0271; A01K 2207/12; A01K 2207/15; A01K 2227/105; A01K 2267/03

(57) ABSTRACT

Provided are non-human animals, including humanized bone marrow/liver/thymus (BLT) non-human animals, that include a recipient immunodeficient animal with human thymus tissue and human liver tissue, both implanted under a kidney capsule of the recipient immunodeficient animal, and transplanted hematopoietic stem cells derived from a human liver tissue. Such non-human animals have human thymus tissue and human liver tissue that are autologous with the hematopoietic stem cells derived from the human liver tissue. Methods of making such BLT non-human animals are also provided. Also disclosed herein are human immune system non-human animals and methods of making the same.

### CROSS-REFERENCE TO RELATED APPLICATIONS

This application claims benefit of U.S. Provisional Patent Application Ser. No. 62/899,995, filed Sep. 13, 2019, herein incorporated by reference in its entirety.

### TECHNICAL FIELD

The presently disclosed subject matter relates to methods of making human mouse xenografts. The presently disclosed subject matter further relates to methods of making human immune system mice and bone marrow/liver/thymus (BLT) mice without using fetal tissue.

### BACKGROUND

During embryogenesis, hematopoietic stem cells migrate from the yolk sac to the placenta and the fetal liver (FL) where they mature and expand. Later on, during development (32-36 weeks of gestation) hematopoietic cells migrate to the bone marrow (BM). Humanized BLT mice are considered the gold standard in the field because they provide a bona fide autologous human thymus where T cell progenitors can develop under the contexts of HLA into T cells. BLT mice are prepared using fetal liver derived human hematopoietic cells and autologous human fetal thymus. However, the use of fetal tissue for biomedical research including the construction of humanized mice has been restricted by the US federal government.

What is needed is a viable alternative to produce humanized BLT mice that does not require the use of human fetal tissue. Prior to the instant disclosure such was not possible. Such solutions, and others disclosed herein, are provided by the instant disclosure.

### SUMMARY

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this Summary does not list or suggest all possible combinations of such features.

Provided herein in some embodiments are non-human animals comprising a recipient immunodeficient animal, human thymus tissue and human liver tissue, both implanted under a kidney capsule of the recipient immunodeficient animal, and transplanted hematopoietic stem cells derived from a human liver tissue. In some aspects, the human thymus tissue and human liver tissue are autologous with the hematopoietic stem cells derived from the human liver tissue. In some embodiments, the human thymus tissue can be derived from neonatal human tissue. The human liver tissue can be derived from neonatal human tissue. Notably, the non-human animals are devoid of human fetal tissue.

In some aspects, the human thymus tissue and human liver tissue are depleted of T-cells. In some embodiments, the autologous cells share HLA alleles. In some embodiments, the hematopoietic stem cells derived from human liver tissue comprise autologous CD34+ cells. In some embodiments, the transplanted hematopoietic stem cells derived from human liver tissue are engrafted in bone marrow of the recipient immunodeficient animal. In some aspects, the transplanted hematopoietic stem cells derived from human liver tissue comprise unfractionated cells and/or as enriched CD34⁺ cells.

In some aspects, such non-human animals can further comprise a human thymic organoid formed from the co-implantation of the human thymus tissue and human liver tissue. In some aspects, the recipient immunodeficient animal can be a mouse, rat or pig, wherein the recipient immunodeficient animal has been treated with a sublethal total body irradiation and/or other preconditioning regimen to cause immunodeficiency in the recipient immunodeficient animal.

In some embodiments, the non-human animal is a humanized bone marrow/liver/thymus (BLT) mouse devoid of human fetal tissue and/or cells derived from fetal tissue. In some aspects, the non-human animal has sustained production of human hematopoietic cells in the peripheral blood. In some aspects, the non-human animal produces one or more of human B, myeloid, T and NK cells, each present in one or more of peripheral blood, primary and secondary immune organs, mucosal tissues, and/or effector tissues.

Provided herein in some embodiments are methods of making a humanized bone marrow/liver/thymus (BLT) non-human animal. Such methods can comprise providing a recipient immunodeficient non-human animal, implanting into the recipient immunodeficient non-human animal human thymus tissue and human liver tissue, both implanted under a kidney capsule of the recipient immunodeficient non-human animal, and administering to the recipient immunodeficient non-human animal hematopoietic stem cells derived from a human liver tissue, wherein the human thymus and liver tissues are autologous with the hematopoietic stem cells derived from the human liver tissue. The non-human animal can be a mouse, rat or pig. Providing the recipient immunodeficient animal can comprise treating the animal with sublethal total body irradiation prior to or concomitantly with the implanting and/or administering steps. In some aspects, the human thymus tissue is derived from neonatal human tissue. In some aspects, the human liver tissue is derived from neonatal human tissue. In some aspects, the resultant humanized BLT non-human animal is devoid of human fetal tissue.

In some aspects, the human thymus tissue and human liver tissue are depleted of T-cells. In some aspects, the autologous cells share HLA alleles. In some aspects, the hematopoietic stem cells derived from human liver tissue comprise autologous CD34+ cells. In some aspects, the administered hematopoietic stem cells derived from human liver tissue engraft in bone marrow of the recipient immunodeficient non-human animal. In some aspects, the transplanted hematopoietic stem cells derived from human liver tissue comprise unfractionated cells and/or as enriched CD34⁺ cells. In some aspects, the administering is by intravenous injection.

In some embodiments, the immunodeficient non-human animal further forms a human thymic organoid from the co-implantation of the human thymus tissue and human liver tissue. In some aspects, the resultant humanized BLT non-human animal is devoid of human fetal tissue and/or cells derived from fetal tissue. In some aspects, the resultant humanized BLT non-human animal has sustained production of human hematopoietic cells in the peripheral blood. In some aspects, the resultant humanized BLT non-human animal produces one or more of human B, myeloid, T and NK cells, each present in one or more of peripheral blood, primary and secondary immune organs, mucosal tissues, and/or effector tissues.

Furthermore, in some embodiments, provided herein are non-human animals comprising a recipient immunodeficient animal, wherein the recipient immunodeficient animal has been treated with sublethal total body irradiation or other preconditioning regimen, and transplanted human neonatal liver derived human CD34+ cells. In some aspects, the non-human animal is devoid of human fetal tissue. The transplanted human neonatal liver derived human CD34+ cells is engrafted in bone marrow of the recipient immunodeficient animal. The immunodeficient animal can be a mouse, rat or pig. The non-human animal can be a human immune system mouse devoid of human fetal tissue and/or cells derived from fetal tissue. In some aspects, the non-human animal produces one or more of human B, myeloid, T and NK cells, each present in one or more of peripheral blood, primary and secondary immune organs, mucosal tissues, and/or effector tissues.

Still yet, also provided are methods of making a human immune system non-human animal, the methods comprising providing a recipient immunodeficient non-human animal, and transplanting into the recipient immunodeficient non-human animal human neonatal liver derived human CD34+ cells. The non-human animal can be a mouse, rat primate, or pig. Providing the recipient immunodeficient animal can comprise treating the animal with sublethal total body irradiation, or an alternative preconditioning regimen, prior to or concomitantly with the transplanting step. The resultant human immune system non-human animal can be devoid of human fetal tissue. In some aspects, the resultant human immune system non-human animal has sustained production of human hematopoietic cells in the peripheral blood. In some aspects, the resultant human immune system non-human animal produces one or more of human B, myeloid, T and NK cells, each present in one or more of peripheral blood, primary and secondary immune organs, mucosal tissues, and/or effector tissues.

Provided herein are pharmaceutical compositions comprising any one or more of the following cells generated in the non-human animal of any of claims 1 to 14 and 31 to 36: CD4+ T cells, CD8+ T cells, CD4+ CD8+ double positive T cells, B cells, monocytes, macrophages, natural killer cells or dendritic cells.

Provided herein are methods to treat an immune-related or immune-mediated disorder or disease in a subject, comprising administering to the subject cells generated in the non-human animals disclosed herein.

These and other objects are achieved in whole or in part by the presently disclosed subject matter. Further, objects of the presently disclosed subject matter having been stated above, other objects and advantages of the presently disclosed subject matter will become apparent to those skilled in the art after a study of the following description, Drawings and Examples.

### BRIEF DESCRIPTION OF THE DRAWINGS

The presently disclosed subject matter can be better understood by referring to the following figures. The components in the figures are not necessarily to scale, emphasis instead being placed upon illustrating the principles of the presently disclosed subject matter (often schematically). In the figures, like reference numerals designate corresponding parts throughout the different views. A further understanding of the presently disclosed subject matter can be obtained by reference to an embodiment set forth in the illustrations of the accompanying drawings. Although the illustrated embodiment is merely exemplary of systems for carrying out the presently disclosed subject matter, both the organization and method of operation of the presently disclosed subject matter, in general, together with further objectives and advantages thereof, may be more easily understood by reference to the drawings and the following description. The drawings are not intended to limit the scope of this presently disclosed subject matter, which is set forth with particularity in the claims as appended or as subsequently amended, but merely to clarify and exemplify the presently disclosed subject matter.

For a more complete understanding of the presently disclosed subject matter, reference is now made to the following drawings in which:

FIGS. 1A through 1C provide characterizations of neonatal thymus and liver tissue. FIG. 1A is an image of neonatal thymus and liver tissue. FIG. 1B shows weight (g) of neonatal thymus (n=2) and liver (n=3) tissue samples. The mean and standard error (s.e.m) are indicated with horizontal and vertical lines respectively. FIG. 1C shows an H&E staining of neonatal thymus and liver tissue (scale bars: 500 um (left panels) and 100 um (right panels)).

FIG. 2A illustrates the presence of CD34⁺ cells in neonatal cord blood as analyzed by flow cytometry. FIGS. 2B and 2C illustrate the presence of CD34⁺ cells in neonatal liver with in vivo repopulating potential. The presence of human CD34⁺ cells in human neonatal liver tissue was analyzed by flow cytometry following enzymatic tissue digest (FIG. 2B). Expression of human CD38 on CD34⁺ neonatal cord blood and liver cells was evaluated by flow cytometry (FIGS. 2A and 2B, right panels). The in vivo systemic repopulating activity of the neonatal liver-derived hematopoietic cells was demonstrated by the presence of human CD45⁺ cells in the periphery of transplanted immunodeficient mice (FIG. 2C).

FIGS. 3A through 3C show results of the analysis of human hematopoietic cells in the peripheral blood of neonatal BLT humanized mice. FIG. 3A is a diagram of neonatal BLT humanized mouse construction. FIG. 3B provides levels of human hematopoietic cells (CD45⁺), B cells (CD19⁺), myeloid cells (CD33+), and T cells (CD3⁺) in the peripheral blood of neonatal BLT humanized mice (n=31 mice). The mean and standard error (s.e.m) are indicated with horizontal and vertical lines respectively. FIG. 3C illustrates the presence of human B cells (CD19⁺), T cells (CD3⁺ and CD4⁺ or CD8⁺), monocytes (CD33⁺CD14⁺), NK cells (CD19^{neg}CD3^{neg}CD33^{neg}CD11b^{neg}CD56⁺) and dendritic cells (lineage^{neg} and CD123⁺ [plasmacytoid dendritic cells] or CD11c⁺ [myeloid dendritic cells]) in the peripheral blood of a neonatal BLT mouse.

FIGS. 4A through 4C show the results of the study of human innate and adaptive immune cells present in the tissues of neonatal BLT mice. FIG. 4A displays data of levels of human CD45⁺ cells in the spleen, lymph nodes, bone marrow, liver and lung of neonatal BLT humanized mice (spleen, bone marrow, liver, and lung: n=19 mice, lymph nodes: n=12 mice). The mean and standard error (s.e.m) are indicated with horizontal and vertical lines respectively. FIG. 4B shows the presence of human hematopoietic cells including monocytes/macrophages (CD33⁺CD14⁺), dendritic cells (lineage^{neg} and CD123⁺ [plasmacytoid dendritic cells] or CD11c⁺ [myeloid dendritic cells]), NK cells (CD19^{neg}CD3^{neg}CD33^{neg}CD11b^{neg}CD56⁺), B cells (CD19⁺) and T cells (CD3⁺ and CD4⁺ or CD8⁺) in the spleen, bone marrow, liver and lung of a neonatal BLT mouse. FIG. 4C confirms the presence of human hematopoietic cells including human B cells (CD19⁺) and T cells (CD3⁺ and CD4⁺ or CD8⁺) in the lymph nodes of a neonatal BLT humanized mouse.

FIG. 5 illustrates that the brain of neonatal BLT humanized mice contains human hematopoietic cells. The presence of human hematopoietic cells including B cells (CD19⁺), T cells (CD3⁺ and CD4⁺ or CD8⁺) and monocytes/macrophages (CD33⁺CD14⁺) is confirmed in the brain of a neonatal BLT mouse.

FIG. 6 confirms that human hematopoietic cells are present in the female and male reproductive tract of neonatal BLT mice. FIG. 6 shows the presence of human hematopoietic cells (CD45⁺) in the female reproductive tract and the epididymis, prostate gland, seminal vesicles, testes and penis of the male reproductive tract of neonatal BLT humanized mice.

FIGS. 7A and 7B demonstrate that the gastrointestinal tract of neonatal BLT humanized mice is reconstituted with human hematopoietic cells. FIG. 7A is an image of H&E staining of the large intestine of neonatal BLT mice. FIG. 7B is an immunofluorescence staining for human CD45+ hematopoietic cells (red) in the large intestine of neonatal BLT mice (left panel, 4× image, right panel, 20× image). Nuclei are stained blue.

FIGS. 8A and 8B demonstrate results of human thymopoiesis in the human thymic organoid of neonatal BLT mice. FIG. 8A is an image of H&E staining of a neonatal BLT humanized mouse human thymic organoid (scale bar: 500 um). A Hassall's corpuscle is shown in the inset image. FIG. 8B illustrates data of progressive acquisition of CD3 on the surface of CD45⁺ cells and the presence of human double positive (CD4⁺CD8⁺) and single positive thymocytes (CD4⁺ or CD8⁺) in the human thymic organoid of a neonatal BLT mouse.

### DETAILED DESCRIPTION

The presently disclosed subject matter now will be described more fully hereinafter, in which some, but not all embodiments of the presently disclosed subject matter are described. Indeed, the disclosed subject matter can be embodied in many different forms and should not be construed as limited to the embodiments set forth herein; rather, these embodiments are provided so that this disclosure will satisfy applicable legal requirements.

General Definitions

The terminology used herein is for the purpose of describing particular embodiments only and is not intended to be limiting of the presently disclosed subject matter.

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

In describing the presently disclosed subject matter, it will be understood that a number of techniques and steps are disclosed. Each of these has individual benefit and each can also be used in conjunction with one or more, or in some cases all, of the other disclosed techniques.

Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about,” when referring to a value or to an amount of a composition, dose, sequence identity (e.g., when comparing two or more nucleotide or amino acid sequences), mass, weight, temperature, time, volume, concentration, percentage, etc., is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

The term “comprising”, which is synonymous with “including” “containing” or “characterized by” is inclusive or open-ended and does not exclude additional, unrecited elements or method steps. “Comprising” is a term of art used in claim language which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms.

As used herein, the term “and/or” when used in the context of a listing of entities, refers to the entities being present singly or in combination. Thus, for example, the phrase “A, B, C, and/or D” includes A, B, C, and D individually, but also includes any and all combinations and subcombinations of A, B, C, and D.

As used herein, “significance” or “significant” relates to a statistical analysis of the probability that there is a non-random association between two or more entities. To determine whether or not a relationship is “significant” or has “significance”, statistical manipulations of the data can be performed to calculate a probability, expressed as a “p value”. Those p values that fall below a user-defined cutoff point are regarded as significant. In some embodiments, a p value less than or equal to 0.05, in some embodiments less than 0.01, in some embodiments less than 0.005, and in some embodiments less than 0.001, are regarded as significant. Accordingly, a p value greater than or equal to 0.05 is considered not significant.

Subjects

In some embodiments a subject treated, screened, tested, or from which a sample is taken, is desirably a human subject, although it is to be understood that the principles of the disclosed subject matter indicate that the compositions and methods are effective with respect to invertebrate and to all vertebrate species, including mammals, such as mice and rats, which are intended to be included in the term “subject”. Moreover, a mammal is understood to include any mammalian species in which screening is desirable, particularly agricultural and domestic mammalian species.

The disclosed methods and treatments are particularly useful in the testing, screening and/or treatment of warm-blooded vertebrates. Thus, the presently disclosed subject matter concerns mammals and birds.

More particularly, provided herein is the modification, testing, screening and/or treatment of mammals such as humans, as well as those mammals of importance due to being laboratory animals for modeling human diseases and conditions (such as mice, rats and pigs), endangered (such as Siberian tigers), of economical importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), and horses. Also provided is the treatment of birds, including the treatment of those kinds of birds that are endangered, kept in zoos, as well as fowl, and more particularly domesticated fowl, i.e., poultry, such as turkeys, chickens, ducks, geese, guinea fowl, and the like, as they are also of economical importance to humans.

Formulations

The compositions of the presently disclosed subject matter comprise in some embodiments a composition that includes a pharmaceutically acceptable carrier. Any suitable pharmaceutical formulation can be used to prepare the adenovirus vectors for administration to a subject.

For example, suitable formulations can include aqueous and no n-aqueous sterile injection solutions which can contain anti-oxidants, buffers, bacteriostats, bactericidal antibiotics and solutes which render the formulation isotonic with the bodily fluids of the intended recipient; and aqueous and non-aqueous sterile suspensions which can include suspending agents and thickening agents. The formulations can be presented in unit-dose or multi-dose containers, for example sealed ampoules and vials, and can be stored in a frozen or freeze-dried (lyophilized) condition requiring only the addition of sterile liquid carrier, for example water for injections, immediately prior to use. Some exemplary ingredients are SDS, mannitol or another sugar, and phosphate-buffered saline (PBS).

It should be understood that in addition to the ingredients particularly mentioned above the formulations of this presently disclosed subject matter can include other agents conventional in the art having regard to the type of formulation in question. For example, sterile pyrogen-free aqueous and non-aqueous solutions can be used.

The therapeutic regimens and compositions of the presently disclosed subject matter can be used with additional adjuvants or biological response modifiers including, but not limited to, the cytokines.

Administration

Administration of the compositions of the presently disclosed subject matter can be by any method known to one of ordinary skill in the art, including, but not limited to intravenous administration, intrasynovial administration, transdermal administration, intramuscular administration, subcutaneous administration, topical administration, rectal administration, intravaginal administration, intratumoral administration, oral administration, buccal administration, nasal administration, parenteral administration, inhalation, and insufflation. In some embodiments, suitable methods for administration of a composition of the presently disclosed subject matter include, but are not limited to intravenous. The particular mode of administering a composition of the presently disclosed subject matter depends on various factors, including the distribution and abundance of cells to be treated, additional tissue- or cell-targeting features of the composition, and mechanisms for metabolism or removal of the composition from its site of administration.

Dosage

An effective dose of a composition of the presently disclosed subject matter is administered to a subject in need thereof. A “therapeutically effective amount” is an amount of the composition sufficient to produce a measurable response (e.g., transplantation of hematopoietic stem cells).

Actual dosage levels of active ingredients in the compositions of the presently disclosed subject matter can be varied so as to administer an amount of the active compound(s) that is effective to achieve the desired therapeutic response for a particular subject. The selected dosage level can depend upon the activity of the therapeutic composition, the route of administration, combination with other drugs or treatments, the severity of the condition being treated, and the condition and prior medical history of the subject being treated. However, it is within the skill of the art to start doses of the compositions at levels lower than required to achieve the desired therapeutic effect and to gradually increase the dosage until the desired effect is achieved.

After review of the disclosure of the presently disclosed subject matter presented herein, one of ordinary skill in the art can tailor the dosages to an individual patient, taking into account the particular formulation, method for administration to be used with the composition, and severity of the condition. Further calculations of dose can consider patient height and weight, severity and stage of symptoms, and the presence of additional deleterious physical conditions. Such adjustments or variations, as well as evaluation of when and how to make such adjustments or variations, are well known to those of ordinary skill in the art of medicine.

Production of Humanized BLT Mice & Human Immune System Mice

The present disclosure is based, at least in part, on the surprising discovery that cells present in the liver from neonates contain human cells with hematopoietic repopulation potential capable of engrafting the bone marrow of mice and producing human progenitor cells that can generate all hematopoietic lineages evaluated to date. In addition, it was discovered that co-implantation of autologous human liver and thymic tissue results in the formation of a human thymic organoid. Therefore, for the first time, the presently disclosed methods provide a viable alternative to the use of fetal tissue for the effective construction and production of humanized BLT mice.

Prior to the instant disclosure, the ability to generate humanized BLT mice with an autologous immune system was based exclusively on the use of human fetal (as opposed to neonatal) liver tissue to obtain hematopoietic stem cells for transplantation, coupled with the use of a combination of human fetal thymic and liver tissue to create human thymic organs after implantation under the kidney capsule.

Now, as disclosed herein, humanized immune system mice and BLT humanized mice have been generated that produce human B, myeloid, T and NK cells, without the use of fetal tissue. The human cells in these reconstituted mice are present in peripheral blood and all tissues analyzed including primary and secondary immune organs (bone marrow/thymus and lymph nodes/spleen respectively), mucosal tissues (gastrointestinal tract, female and male reproductive tract) as well as effector tissues (liver and lung). In addition, the data indicates reconstitution of the mouse brain with human hematopoietic cells.

Thus, in some embodiments, provided herein are non-human animals comprising a recipient immunodeficient animal, wherein the recipient immunodeficient animal is or has been treated with sublethal total body irradiation or an alternative preconditioning regimen sufficient to cause immunodeficiency, and transplanted with human neonatal liver derived human CD34+ cells. In some embodiments, provided herein are non-human animals comprising a recipient immunodeficient animal, wherein the recipient immunodeficient animal is or has been treated with sublethal total body irradiation or an alternative preconditioning regimen to cause immunodeficiency, human thymus tissue and human liver tissue, both implanted under a kidney capsule of the recipient immunodeficient animal, and transplanted hematopoietic stem cells derived from human liver tissue, wherein the human thymus and liver tissues are autologous with the hematopoietic stem cells derived from human liver tissue. In some embodiments, the human thymus tissue is derived from neonatal human tissue. In some embodiments, the human liver tissue is derived from neonatal human tissue. In some embodiments, the non-human animal is devoid of human fetal tissue.

In some aspects, the human thymus tissue and human liver tissue are depleted of T-cells. In some embodiments, the autologous cells share HLA alleles. In some embodiments, the hematopoietic stem cells derived from human liver tissue comprise autologous CD34+ cells. In some embodiments, the transplanted hematopoietic stem cells derived from human liver tissue are engrafted in bone marrow of the recipient immunodeficient animal. In some embodiments, the transplanted hematopoietic stem cells derived from human liver tissue comprise unfractionated cells and/or as enriched CD34⁺ cells.

In some embodiments, the non-human animal further comprises a human thymic organoid formed from the co-implantation of the human thymus tissue and human liver tissue. In some embodiments, the immunodeficient animal is a mouse, rat, primate or pig. In some embodiments, the non-human animal is a humanized bone marrow/liver/thymus (BLT) mouse devoid of human fetal tissue and/or cells derived from fetal tissue. In some embodiments, the non-human animal has sustained production of human hematopoietic cells in the peripheral blood. In some embodiments, the non-human animal produces one or more of human B, myeloid, T and NK cells, each present in one or more of peripheral blood, primary and secondary immune organs, mucosal tissues, and/or effector tissues.

Also provided herein are methods of making a humanized bone marrow/liver/thymus (BLT) non-human animal. In some embodiments, such methods can comprise: (a) providing a recipient immunodeficient non-human animal, (b) transplanting into the recipient immunodeficient non-human animal human thymus tissue and human liver tissue, both transplanted under a kidney capsule of the recipient immunodeficient animal, and (c) administering to the recipient immunodeficient non-human animal hematopoietic stem cells derived from human liver tissue, wherein the human thymus and liver tissues are autologous with the hematopoietic stem cells derived from human liver tissue. In some embodiments, the non-human animal is a mouse, rat or pig. In some embodiments, providing the recipient immunodeficient animal comprises treating the animal with sublethal total body irradiation prior to or concomitantly with step (b) or (c).

In some embodiments, the human thymus tissue is derived from neonatal human tissue. In some embodiments, the human liver tissue is derived from neonatal human tissue. In some embodiments, the resultant humanized BLT non-human animal is devoid of human fetal tissue. In some embodiments, the human thymus tissue and human liver tissue are depleted of T-cells.

In some embodiments, the autologous cells share HLA alleles. In some embodiments, the hematopoietic stem cells derived from human liver tissue comprise autologous CD34+ cells. In some embodiments, the administered hematopoietic stem cells derived from human liver tissue engraft in bone marrow of the recipient immunodeficient non-human animal. In some embodiments, the transplanted hematopoietic stem cells derived from human liver tissue comprise unfractionated cells and/or as enriched CD34⁺ cells. In some embodiments, the administering is by intravenous injection.

In some aspects, the immunodeficient non-human animal further forms a human thymic organoid from the co-implantation of the human thymus tissue and human liver tissue. In some embodiments, the resultant humanized BLT non-human animal is devoid of human fetal tissue and/or cells derived from fetal tissue. In some embodiments, the resultant humanized BLT non-human animal has sustained production of human hematopoietic cells in the peripheral blood. In some embodiments, the resultant humanized BLT non-human animal produces one or more of human B, myeloid, T and NK cells, each present in one or more of peripheral blood, primary and secondary immune organs, mucosal tissues, and/or effector tissues.

As discussed further in the Examples, the neonatal thymus and liver tissue of the disclosed humanized BLT non-human animal models were characterized. Moreover, the data provided herein confirms the presence of CD34⁺ cells in neonatal liver with in vivo repopulating potential. The in vivo systemic repopulating activity of the neonatal liver-derived hematopoietic cells was demonstrated by the presence of human CD45⁺ cells in the periphery of transplanted immunodeficient mice. Additionally, data presented and discussed in the Examples and Figures reveals the presence of human hematopoietic cells in the peripheral blood of neonatal BLT humanized mice, and particularly the presence of human B cells (CD19⁺), T cells (CD3⁺ and CD4⁺ or CD8⁺), monocytes (CD33⁺CD14⁺), NK cells (CD19^{neg}CD3^{neg}CD33^{neg}CD11b^{neg}CD56⁺) and dendritic cells (lineage^{neg} and CD123⁺ [plasmacytoid dendritic cells] or CD11c⁺ [myeloid dendritic cells]) in the peripheral blood of a neonatal BLT mouse.

Moreover, as disclosed herein, the humanized BLT non-human animal models produced with no fetal tissue were shown to have human innate and adaptive immune cells present in various tissues. Particularly, as demonstrated in the Examples and accompanying figures, levels of human CD45⁺ cells were detected in the spleen, lymph nodes, bone marrow, liver and lung of neonatal BLT humanized mice. Additionally, the presence of human hematopoietic cells including monocytes/macrophages (CD33⁺CD14⁺), dendritic cells (lineage^{neg} and CD123⁺ [plasmacytoid dendritic cells] or CD11c⁺ [myeloid dendritic cells]), NK cells (CD19^{neg}CD3^{neg}CD33^{neg}CD11b^{neg}CD56⁺), B cells (CD19⁺) and T cells (CD3⁺ and CD4⁺ or CD8⁺) was confirmed in the spleen, bone marrow, liver and lung of a neonatal BLT mouse.

The present disclosure, including the working Examples and figures, further demonstrates that various tissues of the disclosed humanized BLT non-human animal models, produced with no fetal tissue, contain human hematopoietic cells. For example, the presence of human hematopoietic cells including B cells (CD19⁺), T cells (CD3⁻ and CD4⁺ or CD8⁺) and monocytes/macrophages (CD33⁺ CD14⁺) was confirmed in the brain of a neonatal BLT mouse. Additionally, human hematopoietic cells were present in the female and male reproductive tract of neonatal BLT mice. Finally, by way of example and not limitation, the gastrointestinal tracts of neonatal BLT humanized mice were reconstituted with human hematopoietic cells.

Notably, the present disclosure confirms for the first time the successful production of humanized BLT non-human animal models, produced with no fetal tissue, that demonstrate human thymopoiesis in the human thymic organoid. Such an achievement meets the crucial need to provide humanized BLT animal models that do not require the use of fetal tissue.

Finally, provided herein in some aspects are pharmaceutical compositions comprising any one or more of the following cells generated in the non-human animals disclosed herein: CD4+ T cells, CD8+ T cells, CD4′ CD8+ double positive T cells, B cells, monocytes, macrophages, natural killer or dendritic cells. Correspondingly, also provided herein are methods to treat an immune-related or immune-mediated disorder or disease in a subject, comprising administering to a subject cells generated in the animal models disclosed herein.

### EXAMPLES

The following Examples are included to further illustrate various embodiments of the presently disclosed subject matter. However, those of ordinary skill in the art should, in light of the present disclosure, appreciate that many changes can be made in the specific embodiments which are disclosed and still obtain a like or similar result without departing from the spirit and scope of the presently disclosed subject matter.

### Materials and Methods

Neonatal Cord Blood

Neonatal cord blood was diluted 1:2 in PBS. Mononuclear cells were then enriched by density gradient centrifugation followed by the removal of red blood cells (RBCs) via incubation in an ammonium chloride lysis solution. Following RBC lysis, cells were then centrifuged, resuspended in medium, and counted using trypan blue exclusion. Cells can then be enriched for stem cells (CD34+) using magnetic bead selection. Cord blood cells were then assessed by polychromatic flow cytometry, prepared for HLA genotyping, and then re-suspended in medium for transplantation into mice.

Neonatal Tissue Dissociation

Neonatal tissue, liver and thymus, was visually assessed and its volume, mass, sex, age, color and condition recorded. Liver and thymus are then removed from the collection buffer, divided into an estimated 10 mL total volume of tissue (average of 9 g), and washed with a 4% Amphotericin B, 4% Penicillin-Streptomycin-L-Glutamine, and RPMI or dPBS solution. A small portion of each tissue, liver and thymus, is collected and fixed for IHC analysis. Another portion of each tissue is saved for implantation surgery. The portions of the tissues to be used for implantation are placed in either the Amphotericin B/Penicillin-Streptomycin-L-Glutamine solution or VEGF buffer solution (human-VEGF-165, mouse-VEGF-164, and human-VEGF-C in RPMI) for approximately 2 hours prior to implantation. A portion of the thymus is physically disassociated over a 70 um cell strainer into a single cell suspension for subsequent analysis. The rest of the liver is processed into a single cell suspension. The liver is mechanically dissociated (using scissors and/or scalpels) then digested in a solution consisting of Collagenase D, Collagenase Dispase, and DNase I in RPMI or in a solution consisting of fatty acid free BSA, Selenous acid, Amphotericin B, 1% Penicillin-Streptomycin-L-Glutamine, Collagenase Type IV, and DNase I in RPMI. Following digestion, the cell suspension is filtered across a 70 um cell strainer. Mononuclear cells in the cell suspension can be enriched by density gradient centrifugation followed by the removal of RBCs via incubation in an ammonium chloride lysis solution. Alternatively, cell suspensions can be directly processed for the removal of RBC. Following RBC lysis, cells are then centrifuged, resuspended in medium, and counted using trypan blue exclusion. Cells can then be used for transplantation as total cells or enriched for stem cells (CD34+) using magnetic bead selection. Thymus and liver cells are then assessed by polychromatic flow cytometry, prepared for HLA genotyping, and liver cells (mononuclear cell suspension or enriched CD34+ cells) are re-suspended in medium for transplantation into mice.

Generation of Humanized Mice

NSG mice (NOD.Cg-Prkdc^{scid} II2rg^{tm1Wjl}/SzJ, Stock No. 005557, Jackson Laboratory, Bar Harbor, Me.) between 10 to 16 weeks of age were used. Mice were sub-lethally irradiated before implantation of one 1-2 mm size piece of neonatal liver sandwiched between two pieces of autologous thymus tissue underneath the left kidney capsule. In addition, following conditioning and implantation, mice were injected intravenously with 1-20×10⁶ autologous liver cells. The development of the acquired human immune system was monitored longitudinally in blood using polychromatic flow cytometry analysis. The mice were monitored and maintained for up to 55 weeks post-surgery (>60 weeks old) in a specific-pathogen free facility maintained by the Division of Comparative Medicine at the University of North Carolina at Chapel Hill (UNC-CH). The Institutional Animal Care and Use Committee (IACUC) approved all protocols used for this study.

Harvest For Analysis of In Vivo Reconstitution With Human Neonatal Tissue Derived Progeny Cells.

At necropsy, peripheral blood and tissues (spleen, lymph nodes, bones, human thymic organoid, liver, lung, brain, gastrointestinal tract and male genital tract or female reproductive tract tissues) were collected from mice. To minimize contamination of peripheral red blood cells in tissues, mice were a trans-cardially perfused with PBS prior to tissue collection. Pieces of tissues were fixed for histological analysis. The remainder of the tissues were processed for cell isolation.

Mononuclear Cell Isolation From Tissues

Plasma from peripheral blood was separated by centrifugation and collected for storage at −80° C. The removed plasma was replaced with equal volume of PBS. Whole blood was used for flow cytometric analysis. Red blood cells (RBC) were lysed using a 1× concentration of BD FACS lysing solution. The spleens, bones, lymph nodes, human thymic organoids, lungs, livers, gastrointestinal tracts, reproductive tracts (female or male) and brains of mice were processed for mononuclear cells (MNC). Spleens, lymph nodes, human thymic organoids, and brains were mechanically dissociated through a 70 um cell strainer. Bones were processed with a mortar and pestle prior passing cells over a cell strainer. Livers, lungs, gastrointestinal tracts, and reproductive tracts were digested prior to passing cells over a cell strainer. MNCs were enriched from liver, lungs and brains using a Percoll density gradient. RBCs were lysed with an Ammonium Chloride solution if needed. Cells were washed and counted via trypan blue exclusion. Samples were analyzed by polychromatic flow cytometry.

Polychromatic Flow Cytometry Evaluation

All samples for flow cytometry evaluation were blocked with 10% mouse IgG and, where applicable, 2% purified mouse IgG2aκ prior to the addition of primary antibodies. The antibody panel for the analysis of cells isolated from the neonatal liver included antibodies directed against human CD34, CD38, CD19, CD3, and 7AAD (Table 1). The antibody panel for the longitudinally monitoring of human immune system reconstitution included antibodies directed against human CD3, CD4, CD8, CD19, CD33, and CD45 (Table 2). The presence of human dendritic cells was assessed using antibodies directed against human CD11c, CD123, Lineage Cocktail 1 (Lin-1), and HLA-DR (Table 3). The antibody panel used for the flow cytometric analysis of the tissues (spleen, lymph nodes, bone marrow, liver, lung, brain, female reproductive tract, and male genital tract) at the time of necropsy included antibodies directed against: human CD3, CD4, CD8, CD11b/Mac-1, CD14, CD16, CD19, CD33, CD45, CD56 (Table 4). Non-specific binding was assessed by isotype controls or by fluorescence −1 panels (Table 5). All samples were washed and then fixed with 2% paraformaldehyde. Flow cytometry data was collected on either a BD FACSCanto or a SORP BD LSRFortessa flow cytometer utilizing BD FACSDiva software for collection, and analyzed using BD FACSDiva software. Cells were distinguished by their forward and side scatter profiles (live), and then by human CD45 positivity (where applicable). For the analysis of neonatal liver cells, 7AAD− cells were gated based on their expression of CD3 and CD19; the CD3−/19− cells were gated further according to their expression of CD34 and CD38. For the longitudinal analysis of peripheral blood collected from mice post-transplant, CD45+ cells were gated based on their expression of CD3 and CD19. CD3+/CD19− cells were further gated by CD4 and CD8 expression, CD3−/CD19− cells were further gated by CD33 expression. For the analysis of human dendritic cells, lineage^{neg}/HLA-DR+ cells were gated by their expression of CD11c and CD123. For the analysis of peripheral blood and tissue cells at necropsy, CD45+ cells were gated based on their expression of CD3 and CD19. CD3+/CD19− cells were further gated by CD4 and CD8 expression, CD3−/CD19− cells were further gated by CD14 and/or CD33 expression. CD3−/11b−/19−/33− cells were further gated by CD56 expression.

TABLE 1
Antibodies for Flow Cytometric Analysis

| Target | Fluorophore | Clone | Manufacturer | Catalog | Isotype | Reactivity |
|---|---|---|---|---|---|---|
| CD3 | FITC | HIT3a | BD Bioscience | 555339 | Mouse IgG₂ₐ, _{K} | Human |
| CD38 | PE | HIT2 | BD Bioscience | 555460 | Mouse IgG₁, _{K} | Human |
| 7AAD | (PerCP) | | BD Bioscience | 559925 | | Viability |
| CD19 | PE-Cy7 | SJ5C1 | BD Bioscience | 557835 | Mouse BALB/c | Human |
| | | | | | IgG₁, _{K} | |
| CD34 | APC | 581 | BD Bioscience | 555824 | Mouse IgG₁, _{K} | Human |

TABLE 2
Antibodies for Flow Cytometric Analysis

| Target | Fluorophore | Clone | Manufacturer | Catalog | Isotype | Reactivity |
|---|---|---|---|---|---|---|
| CD3 | FITC | HIT3a | BD Bioscience | 555339 | Mouse IgG₂ₐ, _{K} | Human |
| CD33 | PE | P67.6 | BD Bioscience | 340679 | Mouse BALB/c IgG₁, _{K} | Human |
| CD8 | PerCP | SK1 | BD Bioscience | 347314 | Mouse BALB/c IgG₁, _{K} | Human |
| CD19 | PE-Cy7 | SJ5C1 | BD Bioscience | 557835 | Mouse BALB/c IgG₁, _{K} | Human |
| CD45 | APC | HI30 | BD Bioscience | 555485 | Mouse IgG₁, _{K} | Human |
| CD4 | APC-H7 | RPA-T4 | BD Bioscience | 560158 | Mouse IgG₁, _{K} | Human |

TABLE 3
Antibodies for Flow Cytometric Analysis

| Target | Fluorophore | Clone | Manufacturer | Catalog | Isotype | Reactivity |
|---|---|---|---|---|---|---|
| Lin-1* | FITC | ** | BD Bioscience | 340546 | *** | Human |
| CD123 | PE | 9F5 | BD Bioscience | 340545 | Mouse BALB/c IgG₁, _{K} | Human |
| HLA-DR | PerCP | L243 | BD Bioscience | 347364 | Mouse BALB/c IgG₂ₐ, _{K} | Human |
| CD11c | APC | S-HCL-3 | BD Bioscience | 340544 | Mouse IgG_{2b}, _{K} | Human |

*CD3, CD14, CD16, CD19, CD20, CD56
**SK7, 3G8, SJ25C1, L27, MΦP9, NCAM16.2
***CD3, CD16, CD19, CD20-Mouse IgG1, _{K} or CD14, CD56-Mouse IgG2b, _{K}

TABLE 4
Antibodies for Flow Cytometric Analysis

| Target | Fluorophore | Clone | Manufacturer | Catalog | Isotype | Reactivity |
|---|---|---|---|---|---|---|
| CD14 | FITC | M5E2 | BD Bioscience | 555397 | Mouse IgG₂ₐ, _{K} | Human |
| CD16 | PerCP-Cy5.5 | 3G8 | BD Bioscience | 560717 | Mouse IgG₁, _{K} | Human |
| CD11b/Mac-1 | PE | ICRF44 | BD Bioscience | 555388 | Mouse IgG₁, _{K} | Human |
| CD19 | PE-Cy7 | SJ5C1 | BD Bioscience | 557835 | Mouse BALB/c IgG₁, _{K} | Human |
| CD56 | APC | B159 | BD Bioscience | 555518 | Mouse IgG₁, _{K} | Human |
| CD3 | APC-R700 | UCHT1 | BD Bioscience | 565120 | Mouse BALB/c IgG₁, _{K} | Human |
| CD8 | APC-Cy7 | SK1 | BD Bioscience | 557834 | Mouse BALB/c IgG₁, _{K} | Human |
| CD45 | BV421 | HI30 | BD Bioscience | 563879 | Mouse IgG₁, _{K} | Human |
| CD4 | BV605 | RPA-T4 | BD Bioscience | 562658 | Mouse IgG₁, _{K} | Human |
| CD33 | BV711 | WM53 | BD Bioscience | 563171 | Mouse BALB/c IgG₁, _{K} | Human |

TABLE 5
Isotype controls for non-specific binding

| Target | Fluorophore | Clone | Manufacturer | Catalog | Immunogen |
|---|---|---|---|---|---|
| Mouse IgG1, κ | PE | MOPC-21 | BD Bioscience | 559320 | mouse myeloma protein |
| Mouse IgG1, κ | APC | MOPC-22 | BD Bioscience | 555751 | mouse myeloma protein |
| Mouse BALB/c IgG2a, κ | FITC | G155-178 | BD Bioscience | 553456 | TNP-keyhole limpet hemocyanin |
| Mouse BALB/c IgG1, κ | PerCP-Cy5.5 | X40 | BD Bioscience | 347212 | Keyhole limpet hemocyanin |

H&E Staining

Fixed tissue pieces were embedded in paraffin, cut into 5 um sections, and mounted onto poly-L-lysine-coated glass slides. Following deparaffinization, slides were incubated in hematoxylin and then counterstained with eosin. Brightfield images were taken using a Nikon Eclipse Ci-L Microscope, Nikon Digital Sight camera, with Nikon's NIS-Elements software. Brightness, contrast and/or white balance were adjusted using Adobe Photoshop.

Immunofluorescent Staining

Fixed, paraffin-embedded 5 um tissue sections were deparaffinization followed by antigen retrieval. Tissue sections were then incubated with a 10% serum solution with 0.1° A Triton X-100 in 1× PBS to block non-specific binding. Tissue sections were then incubated overnight with primary antibodies (CD4 and CD8 for thymic tissue, CD45 for large intestine) at 4° C. followed by incubation with fluorescent conjugated secondary antibodies (Alexa Fluor 488 or Alexa Fluor 568; Table 6). All tissue sections were counterstained with DAPI. Control tissue sections were processed without the primary antibodies to indicate background fluorescence. Fluorescent images were taken using an Olympus BX61 Upright Wide Field Microscope, Hammatsu ORCA RC camera, with Improvision's Velocity software. Images were processed for sharpness/contrast in ImageJ (Fiji v3).

TABLE 6
Antibodies for Immunofluorescent Analysis

| Target | Fluorophore | Clone | Isotype | Reactivity | Manufacturer | Catalog |
|---|---|---|---|---|---|---|
| CD4 | Unconjugated | SP35 | Rabbit | Anti-Human | Genway | GWB-4D5E64 |
| CD8 | Unconjugated | C8/144B | Mouse IgG₁, _{K} | Anti-Human | Invitrogen | MA5-13473 |
| CD45 | Unconjugated | 2B11 + PD7/26 | Mouse IgG₁, _{K} | Anti-Human | Dako | M0701 |
| Anti-rabbit | Alexa Fluor 488 | Polyclonal | Goat IgG | Anti-Rabbit | ThermoFisher Scientific | A-11008 |
| Anti-mouse | Alexa Fluor 568 | Polyclonal | Goat IgG | Anti-Mouse | ThermoFisher Scientific | A-11004 |

### Example 1. Results of Experiments to Develop BLT and Humanized Mice Using Neonatal Tissue

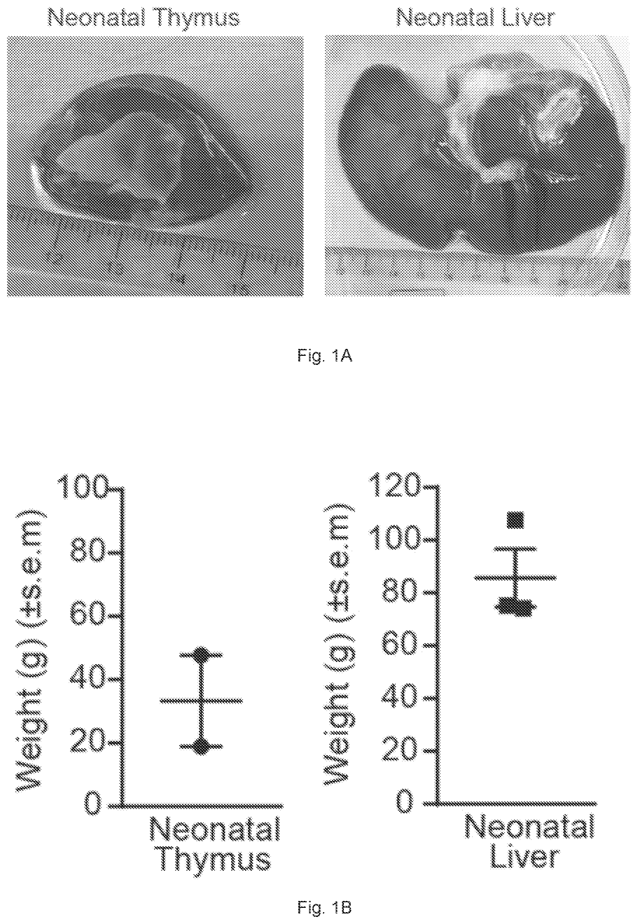

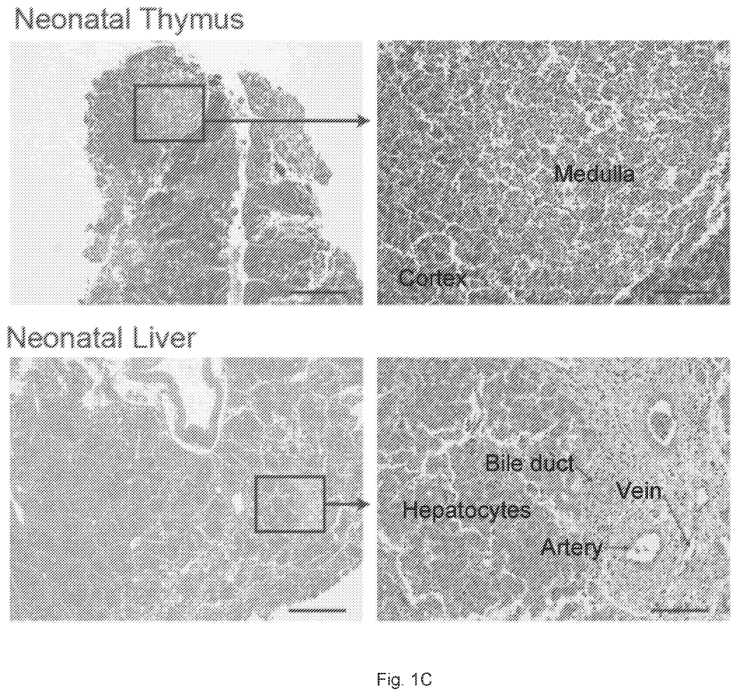

To address the significant obstacles to the development of humanized mice having an autologous human thymus and immune system, for the first time the possibility of using human neonatal tissue and cells was considered and developed. Specifically, cord blood and cadaveric neonatal human thymus and liver tissue was acquired from a non-for profit tissue procurement agency (FIG. 1A). The mean weight of neonatal thymus and liver tissue collected was 33.3 g (range: 18.9 to 47.7 g) and 85.7 g (range: 74.3 to 107.6 g) respectively (FIG. 1B). Neonatal thymus tissue contained an outer cortex that was densely populated with thymocytes and less densely populated inner medullary regions (FIG. 1C). Hepatocytes and liver structures were observed in neonatal liver tissue including bile ducts, hepatic arteries and portal veins (FIG. 1C).

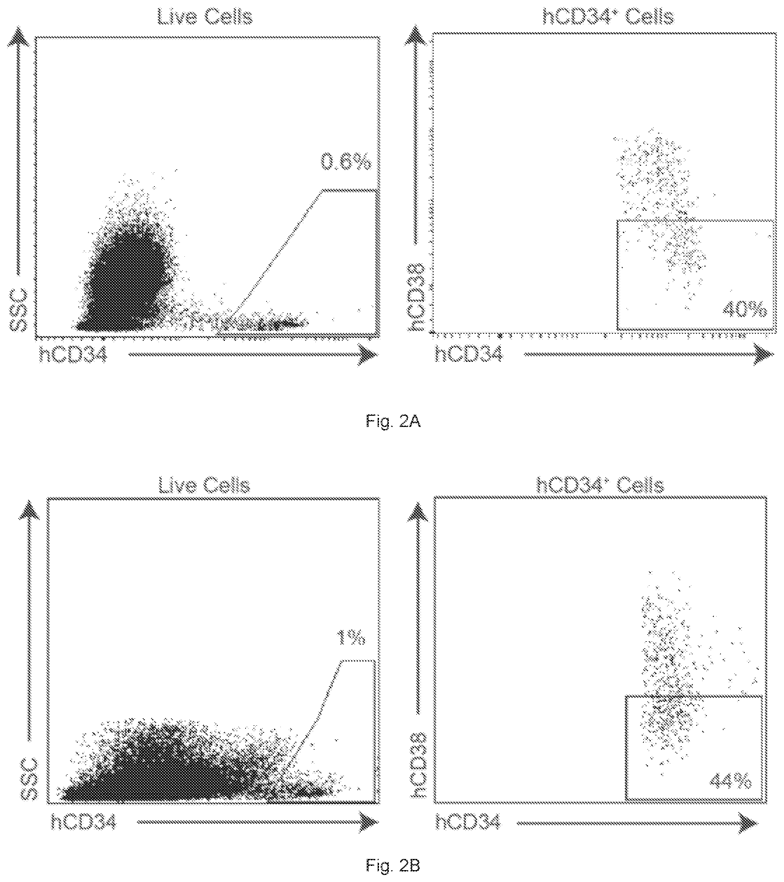

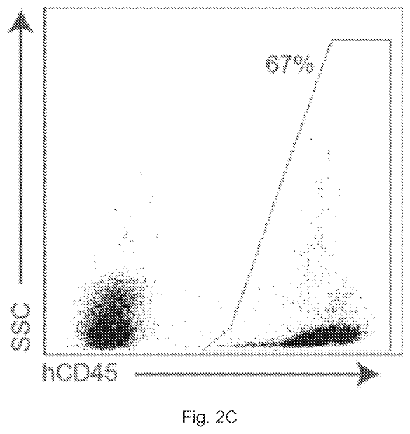

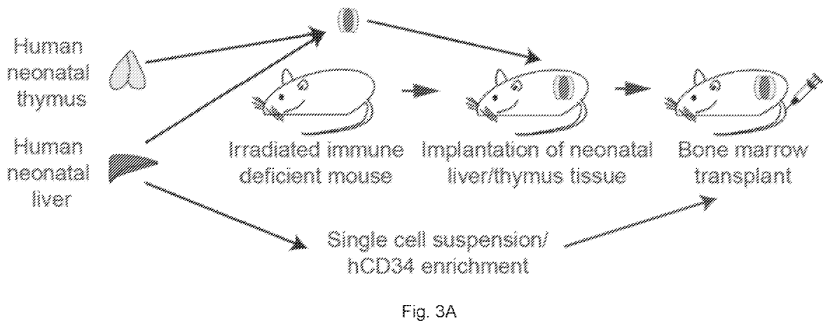

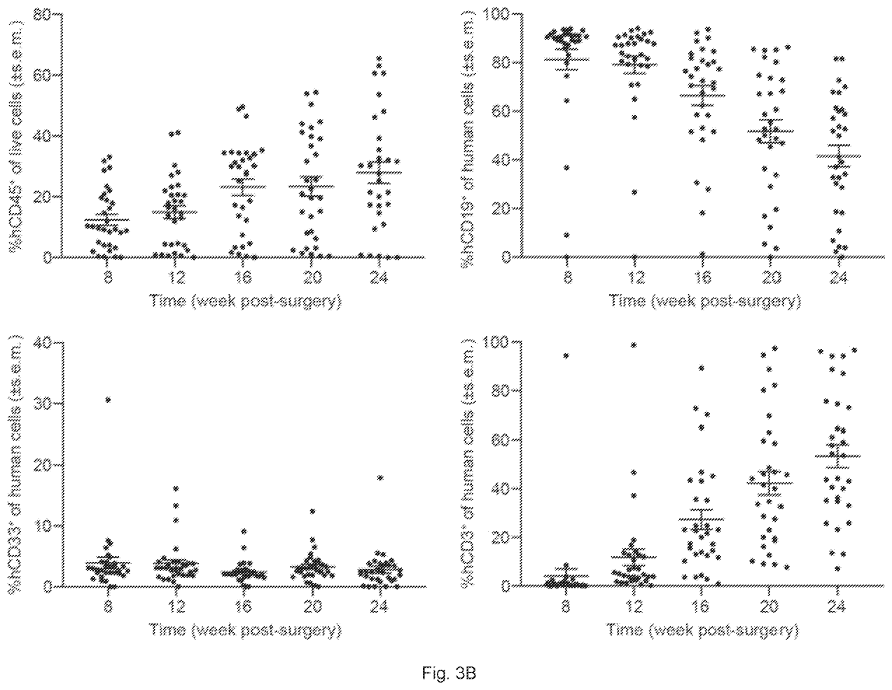

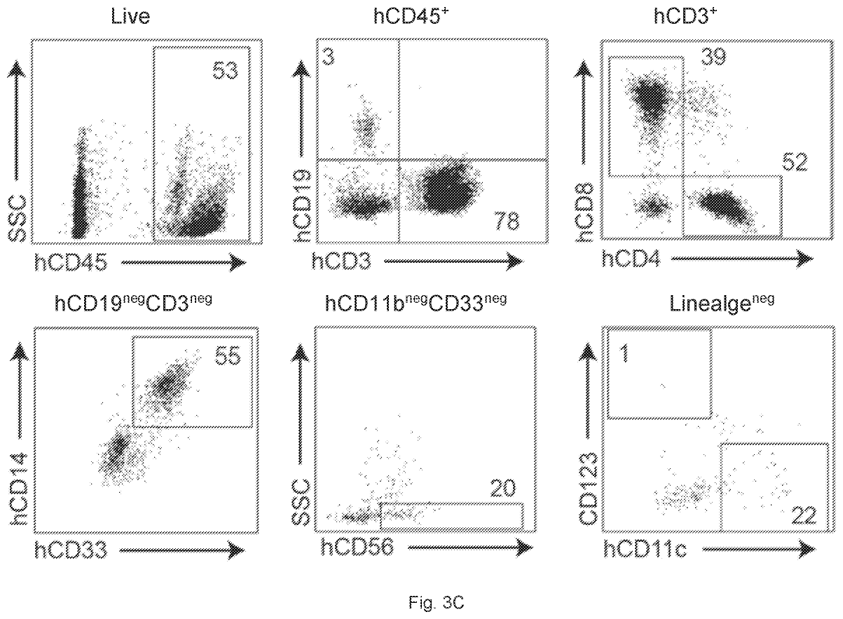

Neonatal cord blood contained human CD34⁺ cells (FIG. 2A) as determined by flow cytometric analysis that can be enriched by magnetic bead selection for transplantation into animals. The thymus and part of the liver were cut into small pieces (1-2 mm³) for implantation under the kidney capsule of immunodeficient mice and the development of the thymus/liver organoid. The rest of the liver was mechanically and enzymatically digested to obtain a single cell suspension. Flow cytometric analysis of these cells demonstrated the presence of human CD34⁺ cells (FIG. 2B). However, the identity of these cells and their in vivo repopulating potential as hematopoietic stem cells could not be determined by flow cytometric analysis. To assess their in vivo repopulating potential, the cells were transplanted into immunodeficient mice either as unfractionated cells or as enriched CD34⁺ cells. Transplantation of the liver cells directly into immunodeficient mice resulted in systemic reconstitution with human hematopoietic cells as demonstrated by the presence of human CD45+ cells in peripheral blood (FIG. 2C). In a separate experiment neonatal liver-derived cells were transplanted into immunodeficient mice that in addition were implanted with thymic tissue that had been incubated ex vivo with growth factors prior to co-implantation with autologous liver tissue under the kidney capsule. Over time human hematopoietic cells expressing the CD45 cell surface marker began to reconstitute the peripheral blood of the transplanted/implanted animals (FIGS. 3A and 3B). Consistent with true hematopoietic cell engraftment, early on the majority of the human cells in the peripheral blood of the transplanted animals were from the B cell (CD19⁺) and myeloid cell (CD33⁺) lineages (FIG. 3B). Over time, human T cells (CD3⁺) were detected in peripheral blood resulting in robust T cell reconstitution (FIG. 3B). These results demonstrated the repopulating potential of the human CD34⁺ cells present in the neonatal liver.

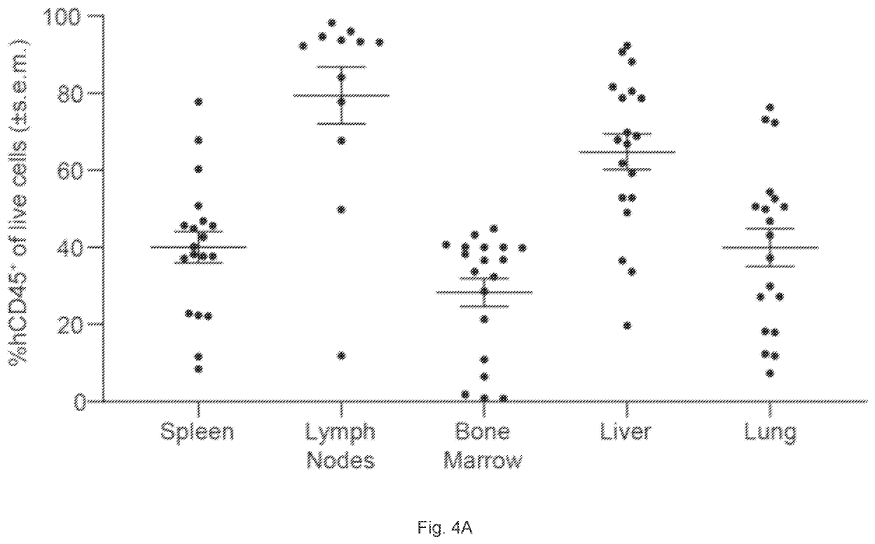

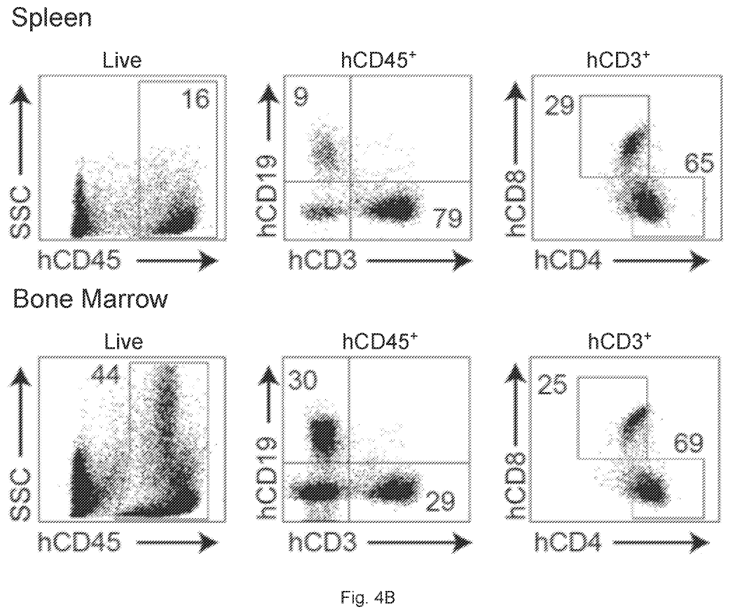

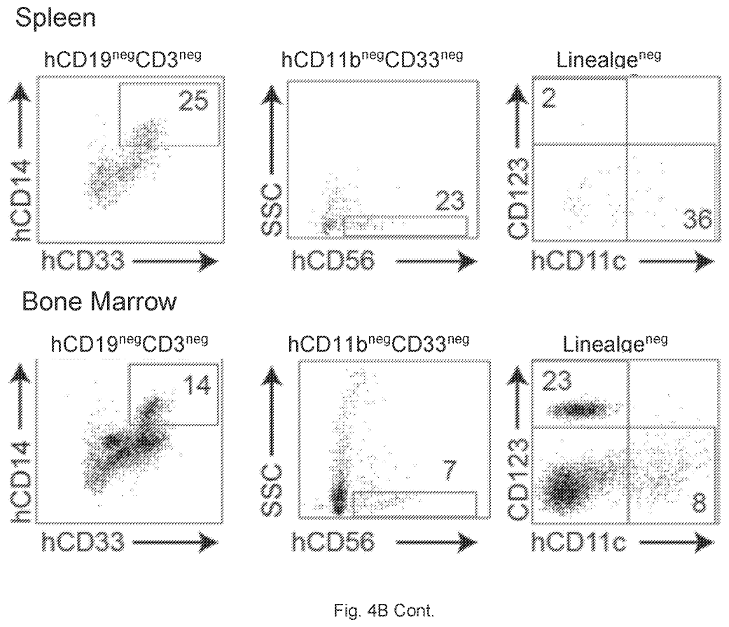

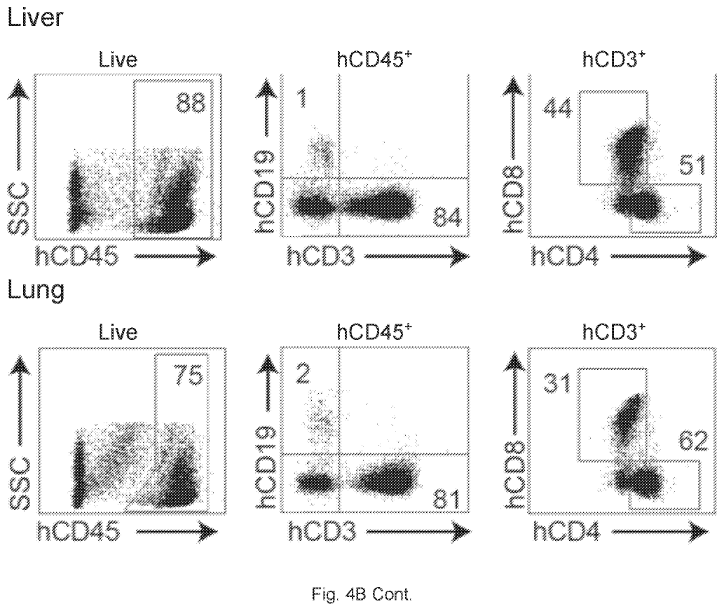

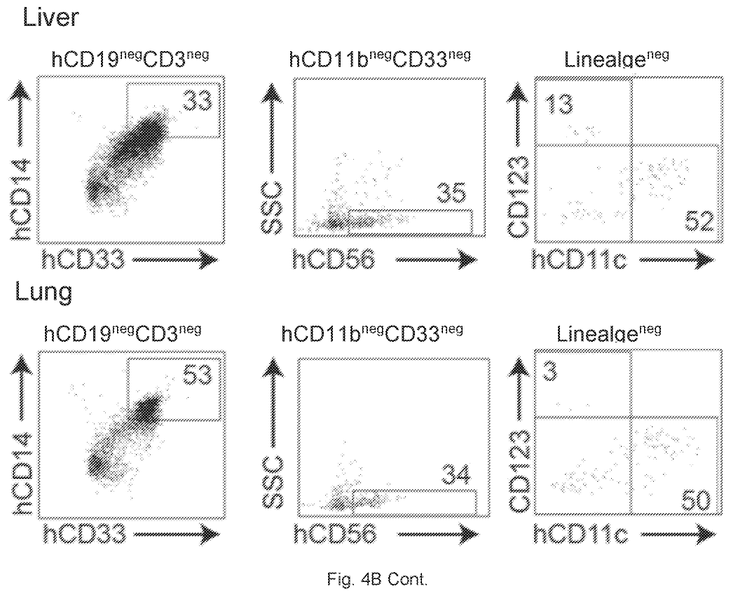

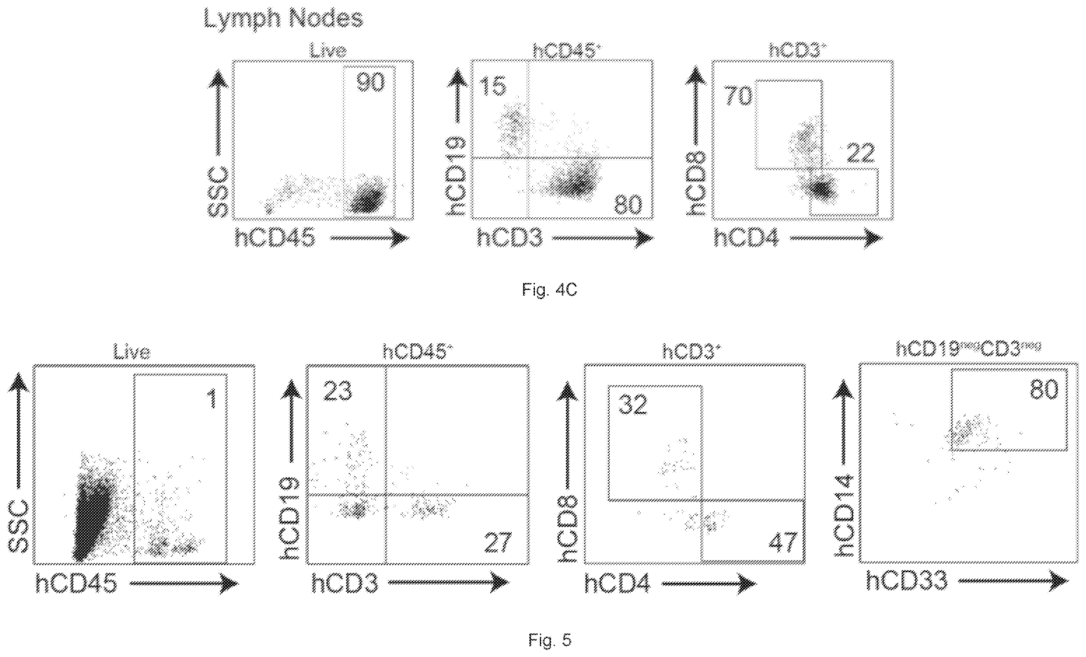

The levels of human cells present in peripheral blood established a steady state level that was maintained for several months (FIG. 3B). Having established sustained production of human hematopoietic cells in the peripheral blood of the implanted/transplanted animals, some animals were harvested to establish whether human hematopoietic cells were also present in tissues. For this purpose, a combination of flow cytometry and immunohistochemical analysis was used. Robust levels of human hematopoietic cells (CD45⁺) were noted in the peripheral blood and tissues of mice including the spleen, bone marrow, liver, and lung (FIGS. 3B, 3C, 4A). The human hematopoietic cell types present included human monocytes/macrophages (CD33⁺CD14⁺), dendritic cells (CD123⁺ plasmacytoid and CD11c⁺ myeloid), natural killer cells (CD56⁺), B cells and T cells (CD4⁺ and CD8⁺ T cells) (FIGS. 3C, 4B). While absent in immunodeficient mice, when transplanted with human neonatal CD34⁺ cells and thymus/liver tissue, animals developed lymph nodes which primarily contained human B cells, CD4⁺ T cells and CD8⁺ T cells (FIG. 4A, 4C).

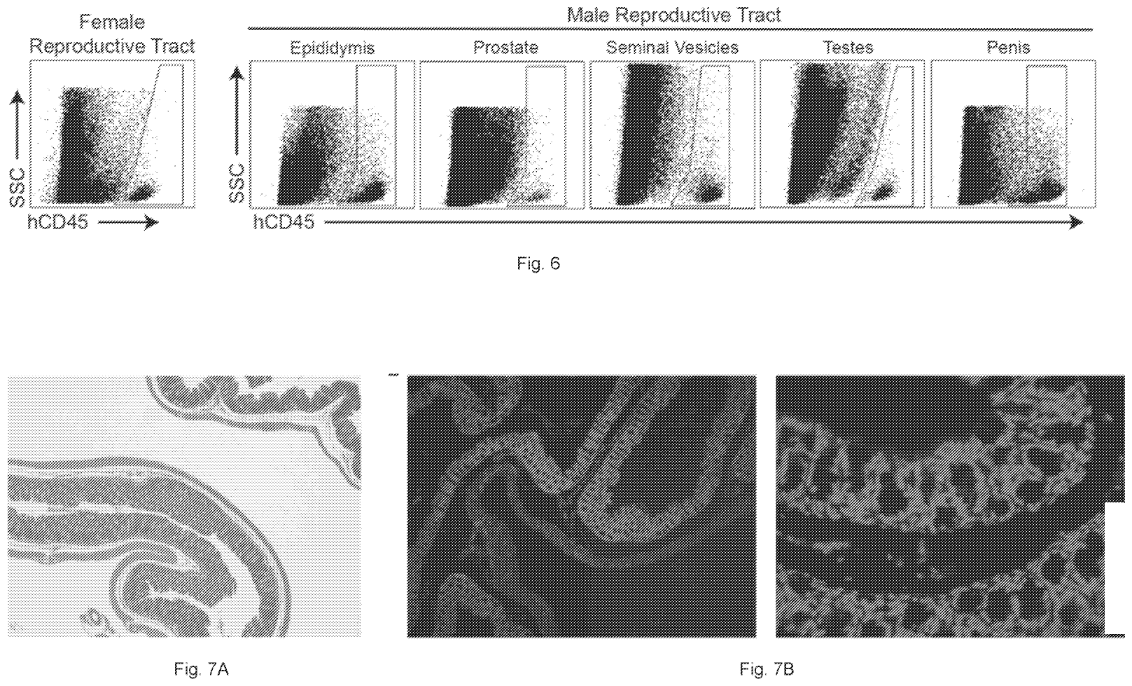

Similar to BLT humanized mice constructed with human fetal tissue/cells, it was also observed that the brain of mice engrafted with neonatal tissue/stem cells was repopulated with human hematopoietic cells (FIG. 5). Specifically, human B cells, T cells (CD4⁺ and CD8⁺) and monocytes/macrophages were present in the brain (FIG. 5). Importantly, the presence of human hematopoietic cells was also observed in key mucosal tissues like the reproductive tract and gastrointestinal tract of these mice. Human CD45⁺ cells were observed in the female reproductive tract and male reproductive tract components including the epididymis, prostate gland, seminal vesicles, testes and penis (FIG. 6). Immunohistochemical analysis of the gastrointestinal tract also revealed the presence of human CD45⁺ cells (FIG. 7).

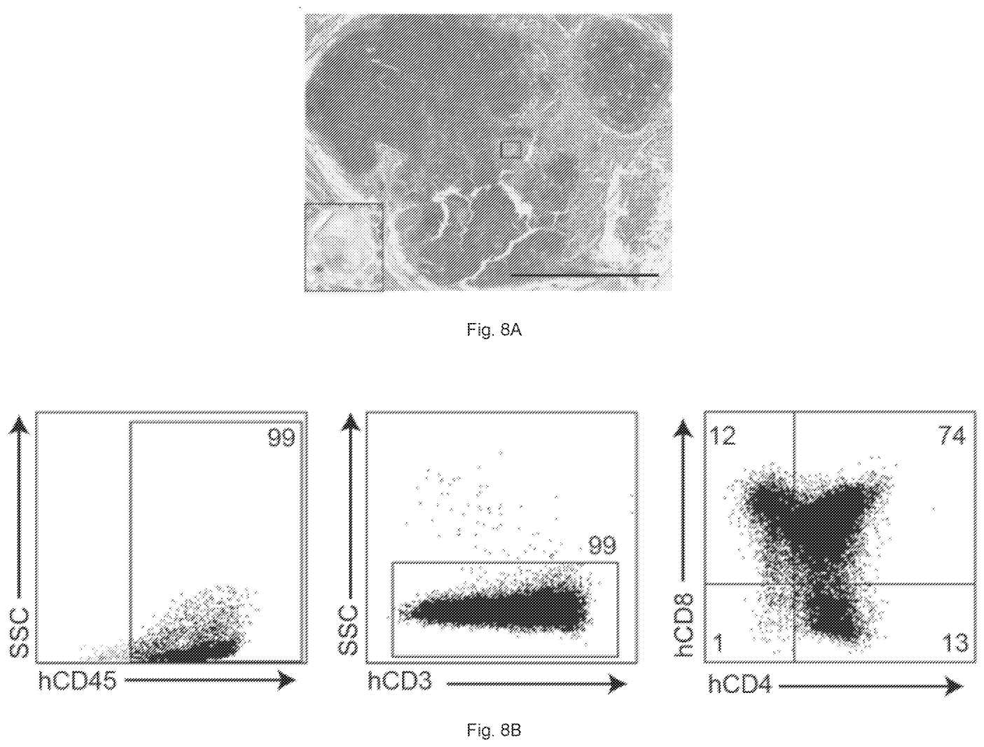

In the implanted thymic tissue, the presence of structures that are characteristic of the human thymus (e.g. Hassall's corpuscles) and human thymocytes at characteristic stages of differentiation were identified (i.e. CD4CD8 double negative, CD4 or CD8 single positive and CD4CD8 double positive) (FIG. 8). Together these results demonstrate the ability of the human cells present in the liver to efficiently repopulate the rodent host and the human neonatal liver/thymus implant to produce human T cells recapitulating key aspects of the use of fetal tissue in the creation of BLT humanized mice.

An example protocol for producing humanized BLT mice using neonatal tissues (non-fetal tissues) is provided herein. It will be understood that this example protocol is for exemplary purposes only and is not intended to be limiting.

### Example 2. Discussion of Results

These results show for the first time that transplantation of human CD34⁺ cells isolated from neonatal liver into immunodeficient mice have in vivo hematopoietic repopulation potential. In addition, when transplanted into animals previously implanted with autologous human thymic and liver tissue, the results obtained mimics those obtained when animals are prepared in the same manner but using fetal liver and thymic tissue. Specifically, these animals have long-term, systemic reconstitution with human hematopoietic cells in the peripheral blood and all tissues analyzed including the spleen, bone marrow, lymph nodes, lung, liver, gastrointestinal tract, reproductive tract (female and male), and brain. Mice were repopulated with human innate and adaptive immune cells including monocytes, macrophages, dendritic cells, natural killer cells, B cells, and T cells. Lymph nodes were also formed in transplanted/implanted animals. Importantly, the presence of human CD4CD8 double negative, CD4CD8 double positive, and CD4 or CD8 single positive thymocytes in implanted human thymus of mice indicative of human T cell development was also observed.

The ability to generate a human immune system and to implant autologous human tissue is not limited to human hematopoietic cells and thymus/liver tissue. Rather, it will extend to all other organs where stem cells have been already discovered (e.g. cord blood and bone marrow) or will be discovered like the human kidney, lung, intestine, heart, etc. In addition, other human tissues can be implanted into these mice alone or in combination to generate new models for biomedical research including the spleen, lymph nodes, lung, kidney, heart, etc.

The applicability of these models for biomedical research includes the evaluation and testing of new or known drugs, biologicals, vaccines, transplant strategies, immunotherapeutics, etc. In addition, these models could be used to expand tissues or cell types in vivo, as vessels for the replication of human or animal pathogens, and to identify new biological or chemical treatments.

It will be understood that various details of the presently disclosed subject matter may be changed without departing from the scope of the presently disclosed subject matter. Furthermore, the foregoing description is for the purpose of illustration only, and not for the purpose of limitation.

What is claimed is:

1. A non-human animal comprising:
a recipient immunodeficient animal;
human thymus tissue and human liver tissue, both implanted under a kidney capsule of the recipient immunodeficient animal; and
transplanted hematopoietic stem cells derived from a human liver tissue,
wherein the human thymus tissue and human liver tissue are autologous with the hematopoietic stem cells derived from the human liver tissue.

2. The non-human animal of claim 1, wherein the human thymus tissue is derived from neonatal human tissue.

3. The non-human animal of any of claims 1 to 2, wherein the human liver tissue is derived from neonatal human tissue.

4. The non-human animal of any of claims 1 to 3, wherein the non-human animal is devoid of human fetal tissue.

5. The non-human animal of claim 1, wherein the human thymus tissue and human liver tissue are depleted of T-cells.

6. The non-human animal of claim 1, wherein the autologous cells share HLA alleles.

7. The non-human animal of claim 1, wherein the hematopoietic stem cells derived from human liver tissue comprise autologous CD34+ cells.

8. The non-human animal of claim 1, wherein the transplanted hematopoietic stem cells derived from human liver tissue are engrafted in bone marrow of the recipient immunodeficient animal.

9. The non-human animal of claim 1, wherein the transplanted hematopoietic stem cells derived from human liver tissue comprise unfractionated cells and/or as enriched CD34⁺ cells.

10. The non-human animal of claim 1, further comprising a human thymic organoid formed from the co-implantation of the human thymus tissue and human liver tissue.

11. The non-human animal of any of claims 1 to 10, wherein the recipient immunodeficient animal is a mouse, rat or pig, wherein the recipient immunodeficient animal has been treated with a sublethal total body irradiation and/or other preconditioning regimen to cause immunodeficiency in the recipient immunodeficient animal.

12. The non-human animal of any of claims 1 to 11, wherein the non-human animal is a humanized bone marrow/liver/thymus (BLT) mouse devoid of human fetal tissue and/or cells derived from fetal tissue.

13. The non-human animal of any of claims 1 to 12, wherein the non-human animal has sustained production of human hematopoietic cells in the peripheral blood.

14. The non-human animal of any of claims 1 to 13, wherein the non-human animal produces one or more of human B, myeloid, T and NK cells, each present in one or more of peripheral blood, primary and secondary immune organs, mucosal tissues, and/or effector tissues.

15. A method of making a humanized bone marrow/liver/thymus (BLT) non-human animal, the method comprising:
(a) providing a recipient immunodeficient non-human animal;
(b) implanting into the recipient immunodeficient non-human animal human thymus tissue and human liver tissue, both implanted under a kidney capsule of the recipient immunodeficient non-human animal; and
(c) administering to the recipient immunodeficient non-human animal hematopoietic stem cells derived from a human liver tissue,
wherein the human thymus and liver tissues are autologous with the hematopoietic stem cells derived from the human liver tissue.

16. The method of claim 15, wherein the non-human animal is a mouse, rat or pig.

17. The method of any of claims 15 to 16, wherein providing the recipient immunodeficient animal comprising treating the animal with sublethal total body irradiation prior to or concomitantly with step (b) or (c).

18. The method of any of claims 15 to 17, wherein the human thymus tissue is derived from neonatal human tissue.

19. The method of any of claims 15 to 18, wherein the human liver tissue is derived from neonatal human tissue.

20. The method of any of claims 15 to 19, wherein the resultant humanized BLT non-human animal is devoid of human fetal tissue.

21. The method of any of claims 15 to 20, wherein the human thymus tissue and human liver tissue are depleted of T-cells.

22. The method of any of claims 15 to 21, wherein the autologous cells share HLA alleles.

23. The method of any of claims 15 to 22, wherein the hematopoietic stem cells derived from human liver tissue comprise autologous CD34+ cells.

24. The method of any of claims 15 to 23, wherein the administered hematopoietic stem cells derived from human liver tissue engraft in bone marrow of the recipient immunodeficient non-human animal.

25. The method of any of claims 15 to 24, wherein the transplanted hematopoietic stem cells derived from human liver tissue comprise unfractionated cells and/or as enriched CD34⁺ cells.

26. The method of any of claims 15 to 25, wherein the administering is by intravenous injection.

27. The method of any of claims 15 to 26, wherein the immunodeficient non-human animal further forms a human thymic organoid from the co-implantation of the human thymus tissue and human liver tissue.

28. The method of any of claims 15 to 27, wherein the resultant humanized BLT non-human animal is devoid of human fetal tissue and/or cells derived from fetal tissue.

29. The method of any of claims 15 to 28, wherein the resultant humanized BLT non-human animal has sustained production of human hematopoietic cells in the peripheral blood.

30. The method of any of claims 15 to 29, wherein the resultant humanized BLT non-human animal produces one or more of human B, myeloid, T and NK cells, each present in one or more of peripheral blood, primary and secondary immune organs, mucosal tissues, and/or effector tissues.

31. A non-human animal comprising:
a recipient immunodeficient animal, wherein the recipient immunodeficient animal has been treated with sublethal total body irradiation or other preconditioning regimen; and
transplanted human neonatal liver derived human CD34+ cells.

32. The non-human animal of claim 31, wherein the non-human animal is devoid of human fetal tissue.

33. The non-human animal of any of claims 31 to 32, wherein the transplanted human neonatal liver derived human CD34+ cells is engrafted in bone marrow of the recipient immunodeficient animal.

34. The non-human animal of any of claims 31 to 33, wherein the immunodeficient animal is a mouse, rat or pig.

35. The non-human animal of any of claims 31 to 34, wherein the non-human animal is a human immune system mouse devoid of human fetal tissue and/or cells derived from fetal tissue.

36. The non-human animal of any of claims 31 to 35, wherein the non-human animal produces one or more of human B, myeloid, T and NK cells, each present in one or more of peripheral blood, primary and secondary immune organs, mucosal tissues, and/or effector tissues.

37. A method of making a human immune system non-human animal, the method comprising:
(a) providing a recipient immunodeficient non-human animal; and
(b) transplanting into the recipient immunodeficient non-human animal human neonatal liver derived human CD34+ cells.

38. The method of claim 37, wherein the non-human animal is a mouse, rat primate, or pig.

39. The method of any of claims 37 to 38, wherein providing the recipient immunodeficient animal comprising treating the animal with sublethal total body irradiation, or an alternative preconditioning regimen, prior to or concomitantly with step (b).

40. The method of any of claims 37 to 39, wherein the resultant human immune system non-human animal is devoid of human fetal tissue.

41. The method of any of claims 37 to 40, wherein the resultant human immune system non-human animal has sustained production of human hematopoietic cells in the peripheral blood.

42. The method of any of claims 37 to 41, wherein the resultant human immune system non-human animal produces one or more of human B, myeloid, T and NK cells, each present in one or more of peripheral blood, primary and secondary immune organs, mucosal tissues, and/or effector tissues.

43. A pharmaceutical composition comprising any one or more of the following cells generated in the non-human animal of any of claims 1 to 14 and 31 to 36: CD4+ T cells, CD8+ T cells, CD4+ CD8+ double positive T cells, B cells, monocytes, macrophages, natural killer cells or dendritic cells.

44. A method to treat an immune-related or immune-mediated disorder or disease in a subject, comprising administering to the subject cells generated in the non-human animal of any of claims 1 to 14 and 31 to 36.
